(19) United States
(12) Patent Application Publication
(10) Pub. No.: US 2002/0193441 A1
(43) Pub. Date: Dec. 19, 2002

(54) Prostanoid therapies for the treatment of glaucoma

(76) Inventor: Stella M. Robertson, Fort Worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/080,364

(22) Filed: Feb. 21, 2002

Related U.S. Application Data
(60) Provisional application No. 60/270,228, filed on Feb. 21, 2001.

(51) Int. Cl. A61K 31/557

(52) U.S. Cl. 514/573

(57) ABSTRACT

Compositions and methods for the treatment of glaucoma and/or ocular hypertension in humans utilizing improved doses of certain prostaglandin derivatives and analogs are disclosed.

This application is a continuation-in-part of U.S. Ser. No. 60/270,228, filed Feb. 21, 2001.

### FIELD OF THE INVENTION

The present invention relates to improved methods of treating glaucoma and ocular hypertension using prostaglandin derivatives and analogs.

Glaucoma is a progressive disease which leads to optic nerve damage and, ultimately, total loss of vision. The causes of this disease have been the subject of extensive studies for many years, but are still not fully understood. The principal symptom of and/or risk factor for the disease is elevated intraocular pressure or ocular hypertension due to excess aqueous humor in the anterior chamber of the eye.

The causes of aqueous humor accumulation in the anterior chamber are not fully understood. It is known that elevated intraocular pressure (“IOP”) can be at least partially controlled by administering drugs which either reduce the production of aqueous humor within the eye, such as beta-blockers and carbonic anhydrase inhibitors, or increase the outflow of aqueous humor from the eye, such as miotics, sympathomimetics, and more recently, prostaglandin analogs.

It is well established that glaucoma and blindness resulting therefrom may occur at different rates among different races (Higginbotham et al.; Higginbotham et al. 1998), describing such differences between black and white patients. The prevalence of glaucoma has been shown to be four to five times greater in African-Americans than in other races. Blindness from glaucoma is four to eight times more common in African-Americans than in Caucasian Americans. Overall, glaucoma is more severe in black patients, requires more medications, and progresses faster to require surgical intervention. (Tielsch et al. 1991; Sommer et al. 1991; Leske et al. 1994; Higginbotham et al. 1996).

Prostaglandins are metabolite derivatives of arachidonic acid. Arachidonic acid in the body is converted to prostaglandin G₂, which is subsequently converted to prostaglandin H₂. Other naturally occurring prostaglandins are derivatives of prostaglandin H₂. A number of different types of prostaglandins are known in the art including A, B, C, D, E, F, G, I and J-Series prostaglandins (EP 0 561 073 A1). Of interest in the present invention are prostaglandin derivatives and analogs which are believed to lower IOP without undue side effects. Such compounds are known in the art. For example, U.S. Pat. Nos. 5,151,444; 5,422,368; 5,688,819; and 5,889,052 describe prostaglandin derivatives and analogs said to exhibit reduced side effects and enhanced therapeutic profiles. The contents of the foregoing patents are by this reference incorporated herein.

Various concentrations of therapeutics dosing regimens are known in the art for the topical treatment of glaucoma and ocular hypertension. Applicant is not aware, however, of any teaching in the art describing a glaucoma therapy that would benefit particularly susceptible classes such as individuals of African descent, which will be referred to hereinafter as Blacks or black patients.

Based on the foregoing, a need exists for the development of therapies yielding a more efficacious lowering of IOP in susceptible patient groups, while maintaining an acceptable side effect profile.

It has now unexpectedly been discovered that certain classes of individuals suffering from ocular hypertension and/or glaucoma exhibit dose response profiles with prostanoid therapies that are different from the profiles of other classes. Moreover, it has been found that enhanced IOP lowering efficacy may be achieved in susceptible classes, and particularly Blacks, by modifying the concentration and/or dosing regimen for a prostanoid medicament.

### SUMMARY OF THE INVENTION

The present invention is directed to improved compositions, and methods of use in treating glaucoma and ocular hypertension in humans. More specifically, and in preferred embodiments, the present invention encompasses compositions and methods for treating glaucoma and ocular hypertension in susceptible classes of patients using higher and/or more frequent doses of a prostanoid medicament.

In certain preferred embodiments, the present invention provides an improved method of treating glaucoma and ocular hypertension in a latent responding patient by topical ocular administration of a prostaglandin derivative or analog, the improvement comprising administering the prostaglandin derivative or analog at a dose which substantially exceeds the baseline dose for such prostaglandin analog to thereby achieve greater intraocular pressure reduction in such patient. Typically, the prostaglandin analog is selected from the group consisting of latanoprost, travoprost, unoprostone isopropyl, and bimatoprost.

In preferred embodiments of the invention where the prostaglandin analog is latanoprost, topical ocular administration is generally effected in an ophthalmically acceptable vehicle having a concentration of latanoprost exceeding 0.005%. Preferably, the concentration of latanoprost is from 0.005% to 0.1%. Most preferably, the concentration of latanoprost is from 0.007% to 0.01%.

Where the prostaglandin analog is travoprost, topical ocular administration is typically effected with an ophthalmically acceptable vehicle having a concentration of travoprost of at least 0.004%. Preferably, the concentration of travoprost is from 0.004% to 0.01%.

Where the prostaglandin analog is bimatoprost, topical ocular administration is typically effected with an ophthalmically acceptable vehicle having a concentration of bimatoprost in excess of 0.03%. Preferably, the concentration of bimataprost is from 0.03% to 1%. Most preferably, the concentration of bimatoprost is from 0.05% to 0.1%.

The invention further provides a method of treating glaucoma or ocular hypertension in a latent responding patient, by administering from 1 μg to 10 μg travoprost to a patient. Preferably, the amount of travoprost administered is from 1.25 μg to 5 μg, most preferably, the amount of travoprost administered is 2 μ. In certain preferred embodiments, the travoprost is administered in a composition comprising 0.004% is travoprost, and at least 2 drops of the composition are administered to the patient, the second drop being administered within about five to ten minutes after the first drop and each subsequent drop being administered within about five to ten minutes of the previous drop.

In another preferred embodiment, the invention provides a method of treating glaucoma or ocular hypertension in a latent responding patient, by administering from 1.25 μg to 10 μg latanoprost to a patient. Preferably, the amount of latanoprost administered is from 1.25 μg to 5 μg, most preferably, the amount of latanoprost administered is from 1.25 to 2 μg. In certain preferred aspects, the latanoprost is administered in a composition comprising 0.005% latanoprost, and at least 2 drops of the composition are administered to the patient, the second drop being administered within about five to ten minutes after the first drop and each subsequent drop being administered within about five to ten minutes of the previous drop.

Alternatively, the invention provides a method of treating glaucoma or ocular hypertension in a latent responding patient, by administering from 9 μg to 30 μg bimatoprost to a patient. Preferably, the amount of bimatoprost administered is from 10 μg to 15 μg, most preferably, the amount of bimatoprost administered is from 11 μg to 14 μg. In preferred aspects, the bimatoprost is administered in a composition comprising 0.03% bimatoprost, and at least 2 drops of the composition are administered to the patient, the second drop being administered within about five to ten minutes after the first drop and each subsequent drop being administered within about five to ten minutes of the previous drop.

### DRAWINGS

The drawings form part of the present specification and are included to further demonstrate certain aspects of the present invention. The invention may be better understood by reference to one or more of these drawings in combination with the detailed description of specific embodiments presented herein.

FIG. 1. Illustrates the relationship between log concentration and IOP for black patients versus non-black patients.

FIG. 2. Illustrates the relationship between log concentration and IOP change for black patients versus non-black patients.

FIG. 3. Illustrates the relationship between actual concentration and IOP for black patients versus non-black patients.

FIG. 4. Illustrates the relationship between actual concentration and IOP change for black patients versus non-black patients.

FIG. 5. Sets forth the number of patients and visits for the demographic subgroup analysis of IOP for the twelve month study, the six month study and the nine month study.

FIG. 6. Sets forth the frequency of demographic subgroups analyzed in the demographic subgroup analysis of IOP for the twelve month study, the six month study and the nine month study.

FIG. 7. Sets forth descriptive statistics for IOP in combined data, by demographic subgroup in the demographic subgroup analysis of IOP for the twelve month study, the six month study and the nine month study.

FIG. 8. Sets forth analysis of variance results for IOP and race for blacks and non-blacks given 0.0015% travoprost, pooled across all three studies (the twelve month study, the six month study and the nine month study).

FIG. 9. Sets forth analysis of variance results for IOP and race for blacks and non-blacks given 0.004% travoprost, pooled across all three studies (the twelve month study, the six month study and the nine month study).

FIG. 10. Provides mean IOP comparison of travoprost 0.004% and Timoptic (timolol) 0.5% combined over the twelve month study and the six month study in Caucasian patients.

FIG. 11. Provides mean IOP comparison of travoprost 0.004% and Timoptic (timolol) 0.5% combined over the twelve month study and the six month study in black patients.

FIG. 12. Provides mean IOP comparison of travoprost 0.004% and Timoptic (timolol) 0.5% combined over the twelve month study and the six month study in other (non-black, non-caucasian) patients.

FIG. 13. Provides mean IOP comparison of travoprost 0.004% and Timoptic (timolol) 0.5% for the twelve month study in caucasian patients.

FIG. 14. Provides mean IOP comparison of travoprost 0.004% and Timoptic (timolol) 0.5% for the twelve month study in black patients.

FIG. 15. Provides mean IOP comparison of travoprost 0.004% and Timoptic (timolol) 0.5% for the twelve month study in other (non-black, non-caucasian) patients.

FIG. 16. Provides mean IOP comparison of travoprost 0.004% and Timoptic (timolol) 0.5% for the six month study in caucasian patients.

FIG. 17. Provides mean IOP comparison of travoprost 0.004% and Timoptic (timolol) 0.5% for the six month study in black patients.

FIG. 18. Provides mean IOP comparison of travoprost 0.004% and Timoptic (timolol) 0.5% for the six month study in other (non-black, non-caucasian) patients.

### DETAILED DESCRIPTION OF THE INVENTION

While bound by no theories, it is postulated that the different dose- response profiles discovered to exist among different classes of glaucoma and ocular hypertension patients may be attributable to anatomical or physiological differences in the eyes of such patients. Differences in number, (i.e. density), type and activity of the various prostaglandin receptors in the tissues of the eye may be responsible, but this has not been established. Degree of iris pigmentation may also be a factor, as Blacks, who characteristically have very dark irises, are found to be a class that benefits from the dosing of the present invention. Alternatively, differences in rates of penetration to the receptor due to anatomical differences (blacks are known to have thinner corneas) and/or biochemical differences (the distribution and activity of the acyl-hydrolosis converting prostaglandin analog prodrugs to the active acid species is different for blacks and non-blacks) may be the underlying cause of this observation.

The present discovery was made during clinical trials with travoprost in glaucoma patients. The results from three separate clinical studies (described in Example 1) involving a 0.0015% travoprost composition and a 0.004% travoprost composition administered to black patients and non-black patients were compared. At a dose of 0.0015%, caucasians (non-blacks) and blacks exhibited comparable IOP reductions, but when the dose was increased to 0.004% caucasians saw no further reduction in IOP while blacks experienced a statistically significant further reduction in IOP. In other words, the concentration that achieved maximum response in Caucasians (hereinafter “base-line concentration”) yielded a less than maximal response in blacks. Classes of patients that may achieve further IOP reduction by increasing dose over the minimum dose that yields maximal response in Caucasians will be referred to herein as latent responders. Other potential latent responders may include hispanics, American indians, asians, aboriginees and other dark complected classes.

It was further shown that mean IOP remained lower for blacks than for non-blacks throughout the day, when the two groups were administered the 0.004% travoprost solution. The analyses of the clinical study data were performed for IOP change from baseline IOP to take into account any baseline IOP differences between blacks and non-blacks. At the 0.0015% dose, there was no significant difference in IOP change between the groups. While there appeared to be an interaction between race and visit time, at 0.004% the mean change from baseline in IOP was consistently greater for black than non-black patients. Thus, it is clear that the difference in IOP change between the groups was not due to differences in baseline IOP. Mean IOP changes for black and non-black patients, combined over visit days and times, were −7.1 mmHg for each group when administered the 0.0015% composition. Mean IOP change for black patients administered the 0.004% composition was −8.1 mmHg and was −7.5 mmHg for non-blacks (Table 1).

TABLE 1
IOP Change*

| | Black | Non-Black |
|---|---|---|
| Travoprost 0.0015% | −7.1 | −7.1 |
| Travoprost 0.004% | −8.1 | −7.5 |

*Data pooled over the twelve month study, the six month study and the nine month study through month 12. The 9 a.m. and 11 a.m. IOP values from they nine month study were combined with the 8 a.m. and 10 a.m. IOP values from the twelve month and the six month studies in the pooled analyses. The data is combined over visit and time of day and represents IOP change from baseline (in mmHg). Results are based upon intent-to-treat data sets.

An analysis of race based upon both overall IOP (combined over visit and time of day) and combined IOP (combined over visits) at 8 a.m., 10 a.m., and 4 p.m. demonstrated that black patients had lower mean IOP following treatment with travoprost 0.004% compared to non-blacks (Table 2). The differences in IOP were up to 1.5 mmHg for the combined (8 a.m., 10 a.m., and 4 p.m.) results and was 1.1 mmHg in the overall result.

TABLE 2
Comparison of IOP Based on Race*

| | Black | Non-Black |
|---|---|---|
| Travoprost 0.0015% | 18.6 | 18.7 |
| (Overall) | | |
| (Combined) | | |
| 8 a.m. | 19.6 | 19.4 |
| 10 a.m. | 18.4 | 18.5 |
| 4 p.m. | 17.8 | 18.2 |
| Travoprost 0.004% | 17.4 | 18.5 |
| (Overall) | | |
| (Combined) | | |
| 8 a.m. | 18.6 | 19.3 |
| 10 a.m. | 16.8 | 18.3 |
| 4 p.m. | 16.8 | 18.0 |

*Data pooled over the twelve month study, the six month study and the nine month study through month 12. The 9 a.m. and 11 a.m. IOP values from the nine month study were combined with the 8 a.m. and 10 a.m. IOP values from the twelve month and the six month studies in the pooled analyses. The data is combined over visit and time of day (overall) or combined over visit (combined) and represents mean IOP (in mmHg). Results are based upon intent-to-treat data sets.

It was further observed that mean IOP was significantly greater for blacks administered the 0.0015% composition than for blacks administered the 0.004% composition. Mean IOP for blacks administered the 0.0015% composition, combined over visit days and time, was 18.6 mmHg and that for blacks administered the 0.004% composition was 17.4 mmHg. Mean IOP for non-blacks administered for 0.0015% composition, combined over visit days and time, was 18.7 mmHg and that for non-blacks administered the 0.004% composition was 18.5 mmHg (Table 3).

TABLE 3
Mean IOP*

| | Black | Non-Black |
|---|---|---|
| Travoprost 0.0015% | 18.6 | 18.7 |
| Travoprost 0.004% | 17.4 | 18.5 |

*Data pooled over the twelve month study, the six month study and the nine month study through month 12. The 9 a.m. and 11 a.m. IOP values from they nine month study were combined with the 8 a.m. and 10 a.m. IOP values from the twelve month and the six month studies in the pooled analyses. The data is combined over visit and time of day and represents mean IOP (in mmHg). Results are based upon intent-to-treat data sets.

Because the studies in which the phenomenon was initially observed were predominantly Caucasian, that class is arbitrarily selected to establish baseline dose. Baseline dose for a prostaglandin analog means a dose which yields its maximal or near maximal IOP lowering effect in Caucasians, without appreciable differences in blacks or other races when compared to latanoprost 0.005% solution. By definition, baseline dose for latanoprost is 0.005%.

Doses of the present invention will substantially exceed the baseline dose to achieve a greater IOP reduction in the affected class. With respect to dose, the term “substantially exceed” means a dosing regimen that exceeds the baseline dosing by at least 40% (i.e. 1.4 times the baseline dose), by increasing the amount of the drug administered, whether by increased concentration, volume or frequency of administration. The preferred means of effecting the increased amount used by increasing concentration of the active prostaglandin analog in an ophthalmically acceptable vehicle. Preferred ranges are 1.5 to 10 times baseline, and most preferred is 2-4 times baseline.

Included within the scope of the present invention are the individual enantiomers of the title compounds, as well as their racemic and non-racemic mixtures. The individual enantiomers can be enantioselectively synthesized from the appropriate enantiomerically pure or enriched starting material by means such as those described below. Alternatively, they may be enantioselectively synthesized from racemic/non-racemic or achiral starting materials. (ASYMMETRIC SYNTHESIS 1983-1985; PRINCIPLES OF ASYMMETRIC SYNTHESIS 1996). They may also be isolated from racemic and non-racemic mixtures by a number of known methods, e.g. by purification of a sample by chiral HPLC (A PRACTICAL GUIDE TO CHIRAL SEPARATIONS BY HPLC 1994; CHIRAL SEPARATIONS BY HPLC 1989), or by enantioselective hydrolysis of a carboxylic acid ester sample by an enzyme (Ohno and Otsuka 1989). Those skilled in the art will appreciate that racemic and non-racemic mixtures may be obtained by several means, including without limitation, nonenantioselective synthesis, partial resolution, or even mixing samples having different enantiomeric ratios. Departures may be made from such details within the scope of the accompanying claims without departing from the principles of the invention and without sacrificing its advantages. Also included within the scope of the present invention are the individual isomers substantially free of their respective enantiomers.

As used herein, the terms “pharmaceutically acceptable ester”/“pharmaceutically acceptable cationic salt” means any ester/cationic salt that would be suitable for therapeutic administration to a patient by any conventional means without significant deleterious health consequences; and “ophthalmically acceptable ester”/“ophthalmically acceptable cationic salt” means any pharmaceutically acceptable ester/cationic salt that would be suitable for ophthalmic application, i.e. non-toxic and non-irritating.

Ophthalmic products are typically packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% weight/volume (“% w/v”).

In general, the doses used for the above described purposes will vary, but will be in an effective amount to decrease intraocular pressure and thus treat or improve glaucomatous conditions. As used herein, the term “pharmaceutically effective amount” refers to an amount which lowers intraocular pressure and/or improves the glaucomatous condition in a mammalian, preferably human, patient. When the compositions are dosed topically, they will generally be in a concentration range of from 0.001 to about 1.0% w/v, with 1-2 drops administered once daily for full, or nearly full, agonists, where the drops are administered within five to ten minutes of each other. Partial agonists, such as unoprostone isopropyl, may require 1-2 drops administered at least twice a day.

As used herein, the term “ophthalmically acceptable vehicle” refers to any vehicle which, when formulated, is safe, and provides the appropriate delivery for the topical administration of an effective amount of at least one prostaglandin derivative or analog of the present invention.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Clinical Studies

The ability of travoprost to lower IOP was evaluated in three separate clinical trials, each involving the evaluation of both a low (0.0015%) and a high (0.004%) concentration composition. The studies differed in length, one lasting six months (hereinafter “six month study”), one lasting nine months (hereinafter “nine month study”) and one lasting twelve months (hereinafter “twelve month study”).

Twelve-month Study

The twelve month study was a triple-masked, parallel group, primary therapy study of the safety and efficacy of travoprost 0.0015% and travoprost 0.004% compared to timolol 0.5% and latanoprost 0.005% in patients with open-angle glaucoma or ocular hypertension. The term triple-masked is synonymous with the ICH E3 definition of double-blind. Patients of any race and either sex with open-angle glaucoma (with or without dispersion or pseudoexfoliation component) or ocular hypertension were qualified to participate in the studies.

Pursuant to the study protocol, one group received travoprost 0.0015% topical ocular solution and dropped one drop once-daily in each eye at 8:00 p.m. A second group received travoprost 0.004% topical ocular solution and dropped one drop, once-daily in each eye at 8:00 p.m. A third group received vehicle only (placebo) and dropped one drop, once-daily in each eye at 8:00 a.m. A fourth group received timolol 0.5% topical ocular solution and dropped one drop, twice-daily in each eye at 8:00 a.m. and 8:00 p.m. A fifth group received latanoprost 0.005% topical ocular solution and dropped one drop, once-daily in each eye at 8:00 p.m.

For evaluation purposes, the primary efficacy variable was mean intraocular pressure (IOP) in patient's worse eye. Safety was based on ocular and systemic parameters including hyperemia, flare, iris color, visual acuity, slit-lamp examinations, endothelial cell density, pachymetry, blood pressure and pulse, laboratory assessments, dilated fundus examinations, automated perimetry and adverse events. Repeated measures analysis of variance was used to test differences among treatment groups and to calculate 95% confidence limits about the differences between groups.

Six-month Study

The six month study was a triple-masked, parallel group, primary therapy study of the safety and efficacy of travoprost 0.0015% and travoprost 0.004% compared to timolol 0.5% in patients with open-angle glaucoma or ocular hypertension. The term triple-masked is synonymous with the ICH E3 definition of double-blind. Patients of any race and either sex with open-angle glaucoma (with or without dispersion or pseudoexfoliation component) or ocular hypertension were qualified to participate in the studies.

Pursuant to the study protocol, one group received travoprost 0.0015% topical ocular solution and dropped one drop once-daily in each eye at 8:00 p.m. A second group received travoprost 0.004% topical ocular solution and dropped one drop, once-daily in each eye at 8:00 p.m. A third group received vehicle only (placebo) and dropped one drop, once-daily in each eye at 8:00 a.m. A fourth group received timolol 0.5% topical ocular solution and dropped one drop, twice-daily in each eye at 8:00 a.m. and 8:00 p.m.

For evaluation purposes, the primary efficacy variable was mean intraocular pressure (IOP) in patient's worse eye. Safety was based on ocular and systemic parameters including hyperemia, aqueous cells and flare, iris pigmentation, visual acuity, slit-lamp biomicroscopy, blood pressure and pulse, laboratory assessments, dilated fundus examinations, automated perimetry and adverse events. Repeated measures analysis of variance was used to test differences among treatment groups and to calculate 95% confidence limits about the differences between groups.

Nine-month Study

The nine month study was a triple-masked, parallel group, primary therapy study of the safety and efficacy of travoprost 0.0015% and travoprost 0.004% compared to timolol 0.5% and latanoprost 0.005% in patients with open-angle glaucoma or ocular hypertension. The term triple-masked is synonymous with the ICH E3 definition of double-blind. Patients of any race and either sex with open-angle glaucoma (with or without dispersion or pseudoexfoliation component) or ocular hypertension were qualified to participate in the studies.

Pursuant to the study protocol, one group received travoprost 0.0015% topical ocular solution and dropped one drop once-daily in each eye at 9:00 p.m. A second group received travoprost 0.004% topical ocular solution and dropped one drop, once-daily in each eye at 9:00 p.m. A third group received vehicle only (placebo) and dropped one drop, once-daily in each eye at 9:00 a.m. A fourth group received timolol 0.5% topical ocular solution and dropped one drop, twice-daily in each eye at 9:00 a.m. and 9:00 p.m.

For evaluation purposes, the primary efficacy variable was mean intraocular pressure (IOP) in patient's worse eye. Safety was based on ocular and systemic parameters including hyperemia, aqueous cells and flare, iris color, visual acuity, slit-lamp biomicroscopy, blood pressure and pulse, laboratory assessments, dilated fundus examinations, automated perimetry and adverse events. Repeated measures analysis of variance was used to test differences among treatment groups and to calculate 95% confidence limits about the differences between groups.

### EXAMPLE 2

Comparison of Black v. Non-black Patients

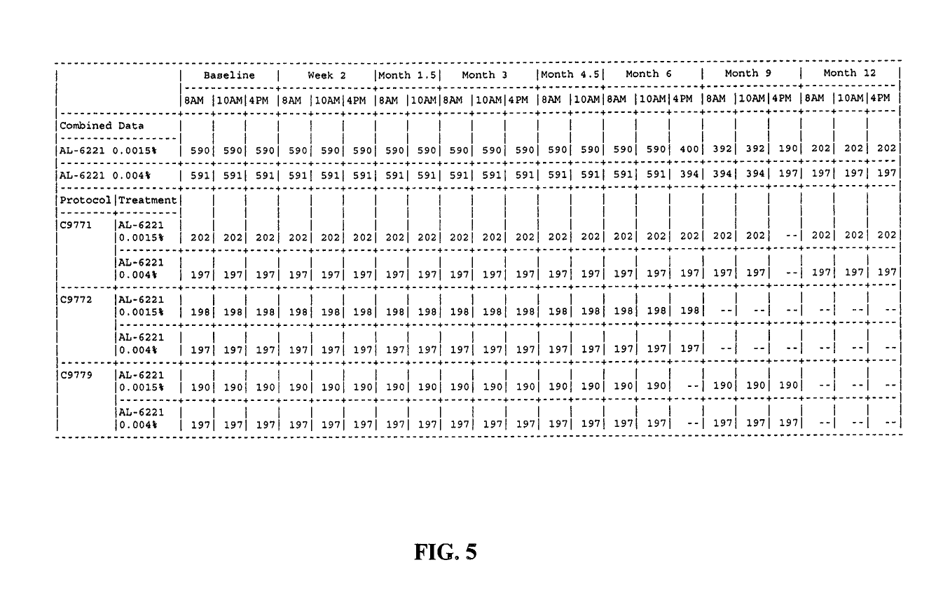

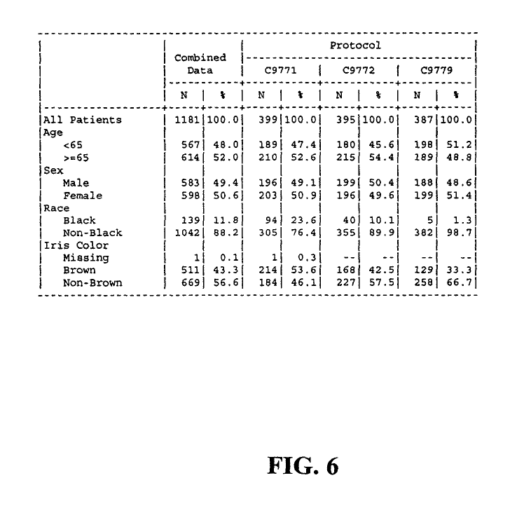

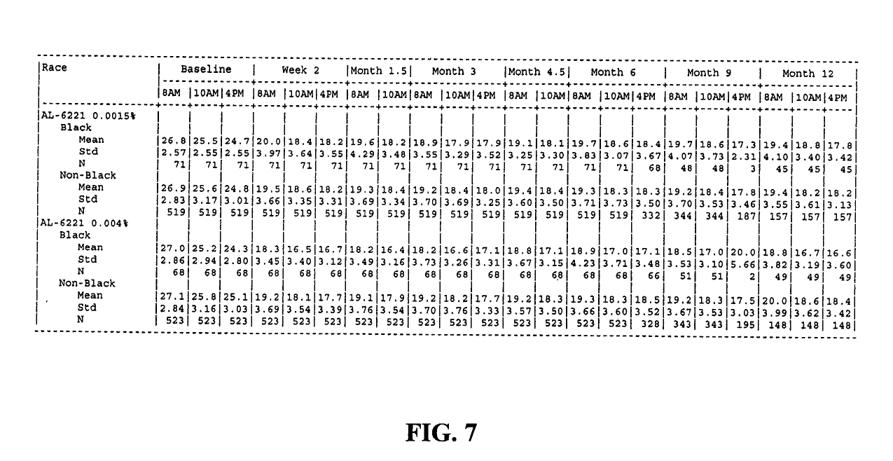

Mean IOP for blacks administered compositions containing 0.0015% travoprost and 0.004% travoprost was compared to that for non-blacks administered the same compositions. The intent-to-treat data were pooled across the three studies described in Example 1. The numbers of patients in the pooled analysis, including the numbers contributed from each protocol, are provided in FIG. 5. Frequencies of the category are provided in FIG. 6. Descriptive statistics are provided in FIG. 7.

Within the subgroup (blacks), comparisons of mean IOP were made using a three-way repeated measures analysis of variance model, with terms for visit day, visit time of day, and their interactions with the subgroup. A random effects term for patient nested within protocol was used to account for repeated measurements.

No statistically significant subgroup differences were observed in the 0.0015% analysis. In the 0.004% analysis, there was a significant subgroup difference in mean IOP between black and non-black patients, in addition to a significant interaction between race and visit time. Although mean IOP was consistently lower for black than non-black patients at 8 AM, 10 AM and 4 PM, the subgroup differences varied as 0.8 mmHg, 1.5 mmHg and 1.1 mmHg, respectively, at these times.

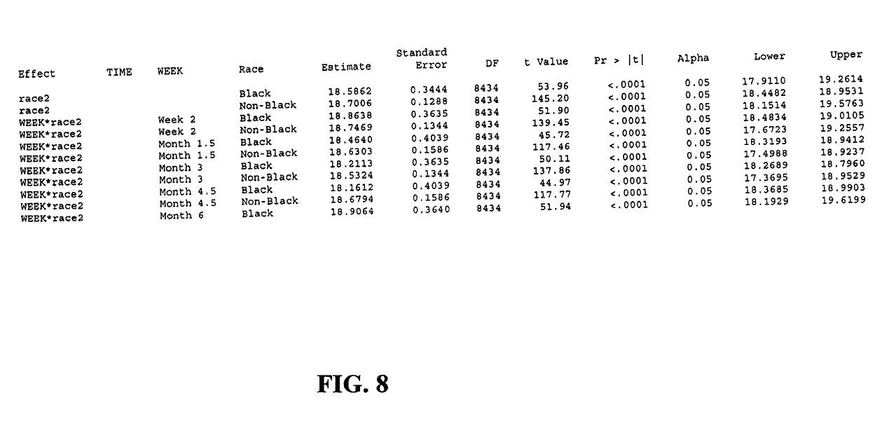

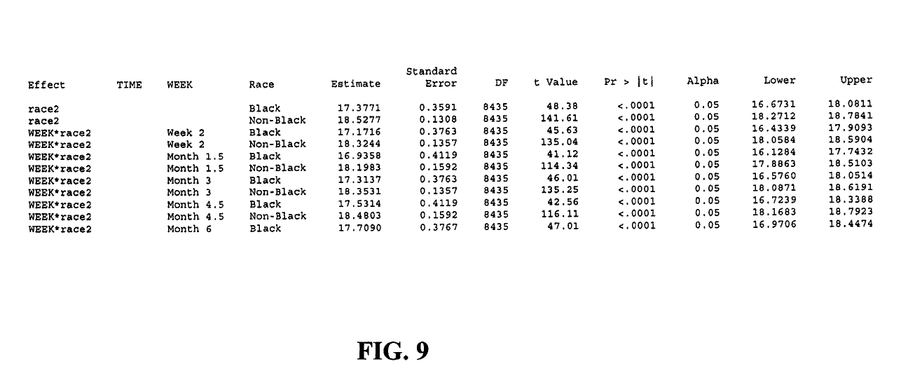

FIGS. 8 and 9 show that mean IOPs for black and non-black patients, combined over visit days and times, were 18.6 and 18.7 mmHg in the 0.0015% travoprost group and 17.4 and 18.5 mmHg in the 0.004% group (Table 3).

### EXAMPLE 3

Dose-response evaluations

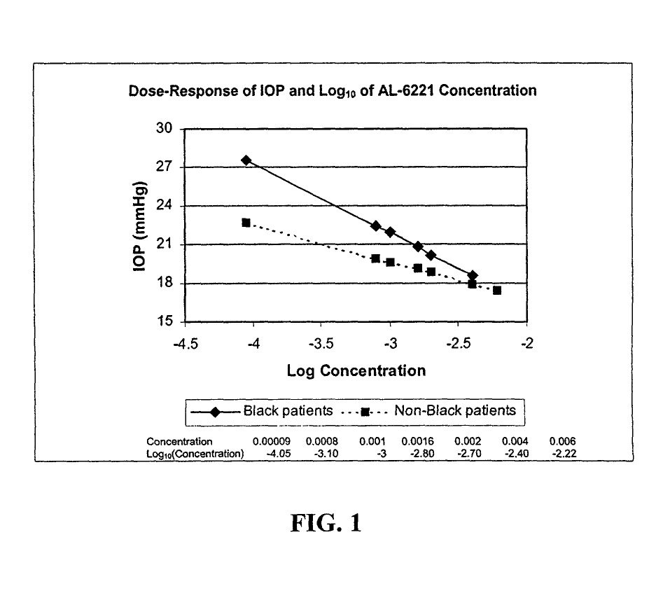

Dose-response studies were undertaken to evaluate the relationship between travoprost and IOP response in black and non-black patients. The results demonstrated that mean IOP was reduced significantly with increasing dose for both race subgroups. However, the data showed a much more pronounced dose-response for black patients than for non-black patients based on comparison slopes. The mean IOP was lowered significantly with increasing dose for both race groups but the black patients exhibited a much more dose dependent relationship. A similar relationship was observed for mean change from baseline IOP and these relationship are graphically represented in FIGS. 1-4.

FIG. 1 depicts the relationship between log concentration and IOP for black patients versus non-black patients. For black patients, IOP went from approximately 28 mmHg at a concentration of 0.00009% travoprost to approximately 18.5 mmHg at 0.004% travoprost. For non-black patients, IOP went from approximately 23 mmHg at 0.00009% travoprost to approximately 18 mmHg at 0.004% travoprost.

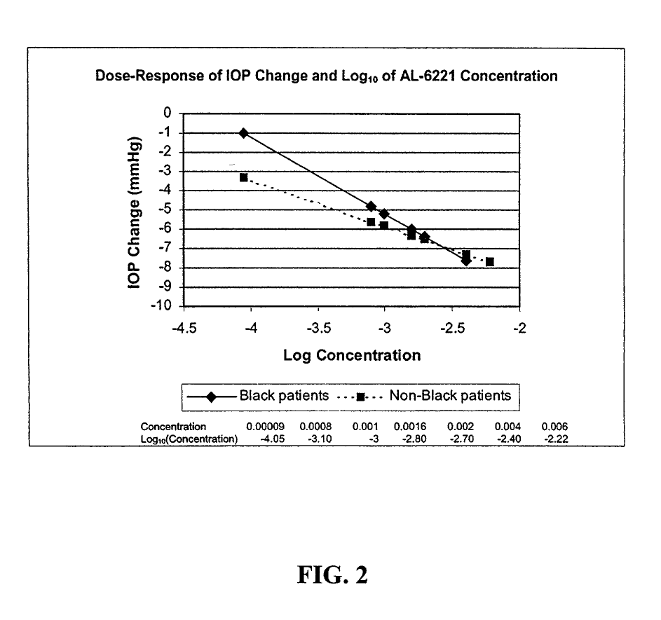

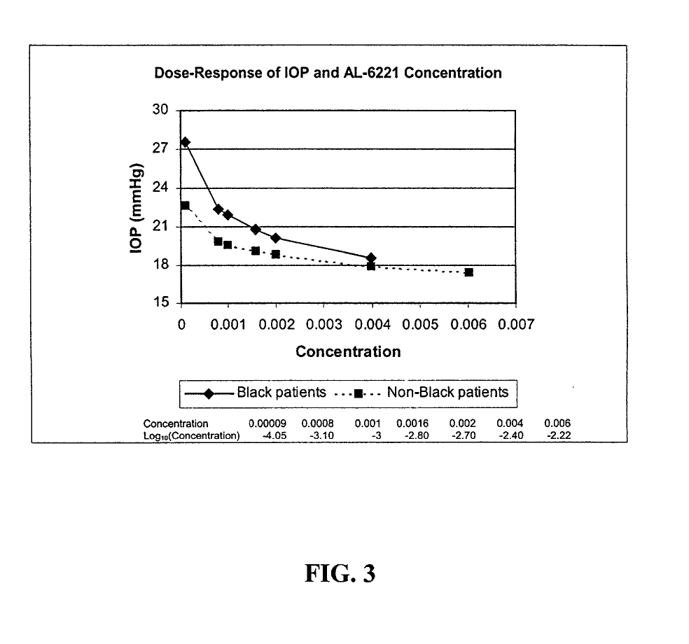

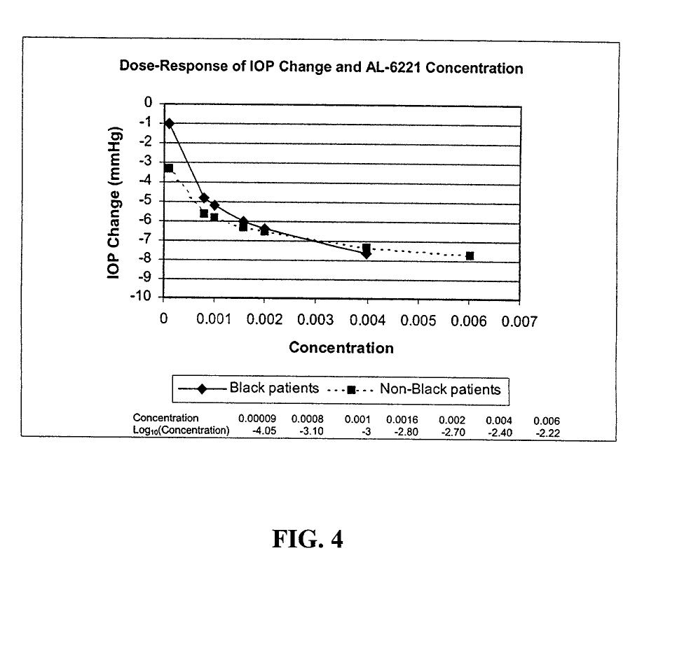

FIG. 2 depicts the relationship between log concentration and IOP change. IOP change went from approximately −1 mmHg to approximately −7.75 mmHg for black patients and from approximately −3.25 mmHg to approximately −7.25 mmHg for non-black patients. FIGS. 1 and 2 depict a comparison of IOP or IOP change to log concentration to illustrate the significance of the dose-response difference between the two groups. FIGS. 3 and 4 depict the same relationships using actual concentrations.

### EXAMPLE 4

Mean IOP Comparisons of Travoprost and Timolol in Caucasian and Black Patients

Model terms for fixed effects were treatment group, visit day, visit time of day, race (Caucasian vs. Black vs. Other), and the consequent two-, three-, and four-factor interactions; and a random effect to take into account repeated observations on a patient. In the single-study analyses, i.e., twelve month study alone and six month study alone, patient nested within treatment was used for the random effect; for the analysis combined over the two clinical studies, patient nested within protocol was used. The treatment comparisons and 95% confidence intervals are based on least squares means derived from the above model.

The baseline and on-therapy estimates were derived from separate analyses of variance. Although not presented, data for patients who received travoprost 0.0015% were included in the models to maintain a covariance structure similar to that used in previous analyses. In the combined analyses of the twelve month and six month studies, patients in the twelve month study who received treatment with latanoprost 0.005% were excluded.

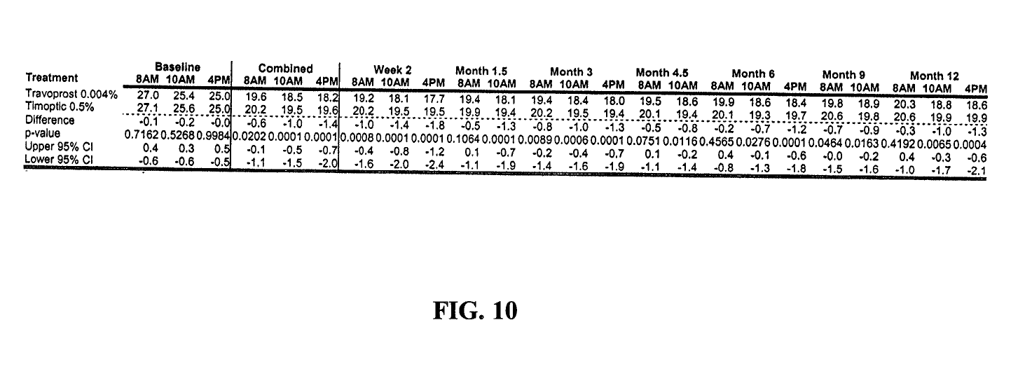

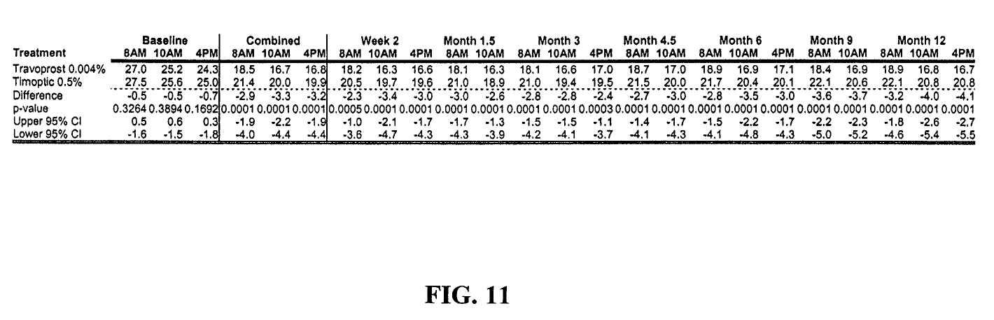

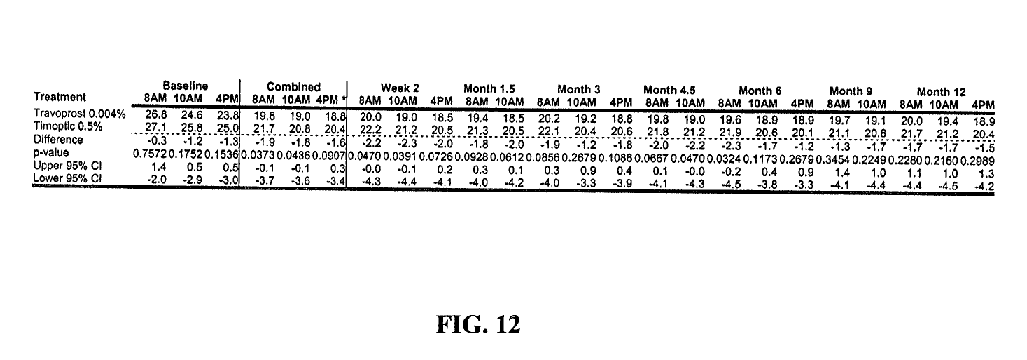

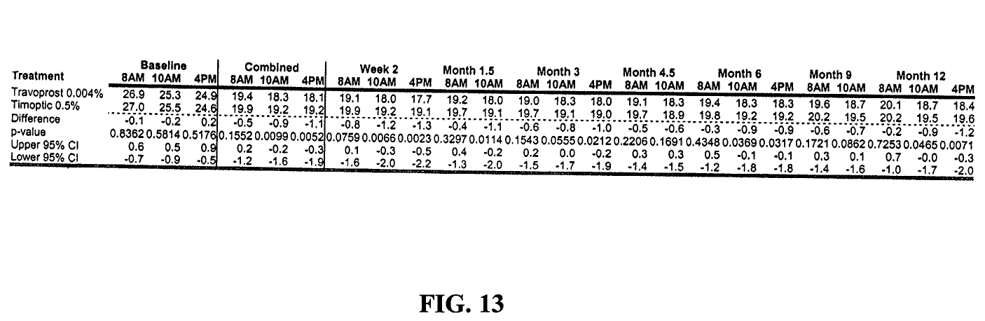

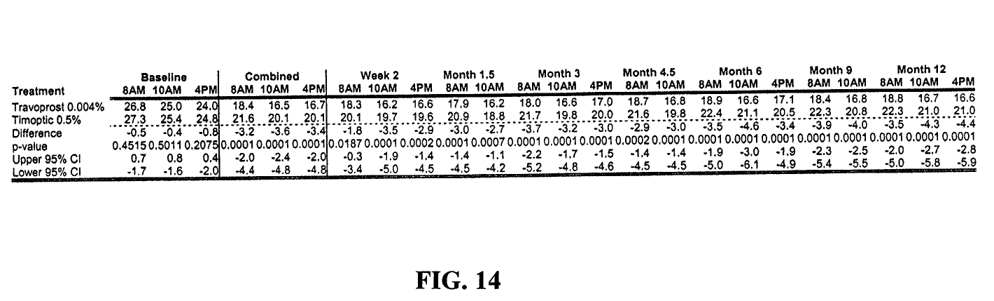

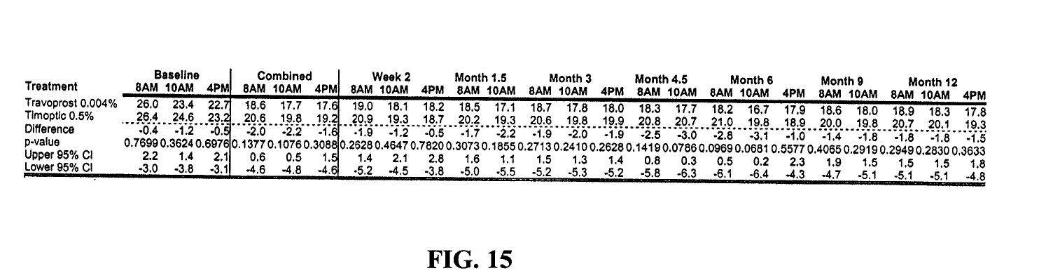

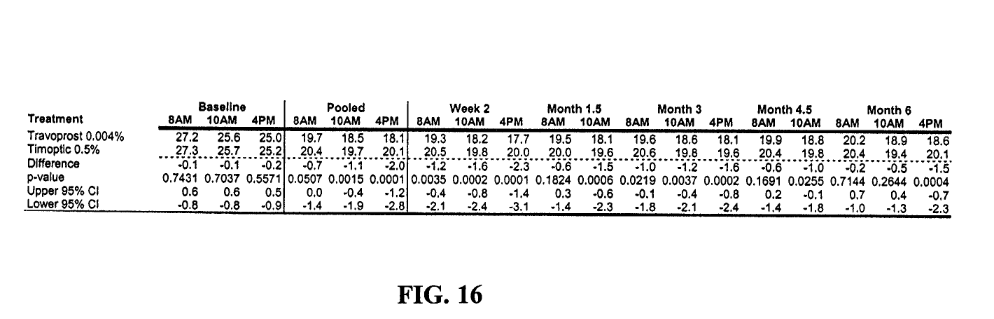

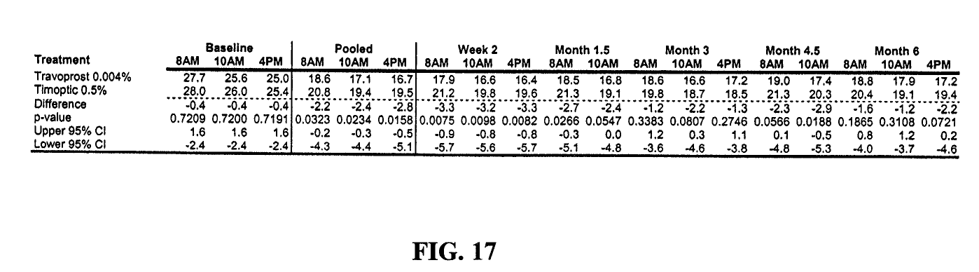

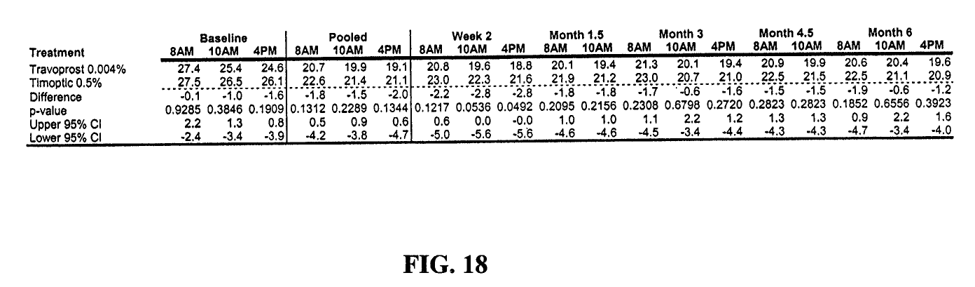

Two-sided 95% confidence intervals were calculated for mean IOP comparisons between travoprost 0.004% and timolol 0.5% combined over the twelve month and six month studies, for the twelve month study alone and for the six month study alone. Results are shown in FIGS. 10-18.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

### References

The following references, to the extent that they provide exemplary procedural or other details supplementary to those set forth herein, are specifically incorporated herein by reference.

U.S. Pat. Nos.

5,151,444

5,422,368

5,688,819

5,889,052

Foreign Patents and Published Applications

EP0561 073A1

Books

A PRACTICAL GUIDE TO CHIRAL SEPARATIONS BY HPLC, G. Subramanian, Ed., VCH Publishers: New York, (1994).

ASYMMETRIC SYNTHESIS, J. D. Morrison and J. W. Scott, Eds., Academic Press Publishers: New York, volumes 1-5 (1983-1985).

CHIRAL SEPARATIONS BY HPLC, A. M. Krstulovic, Ed.; Ellis Horwood Ltd. Publishers, (1989).

PRINCIPLES OF ASYMMETRIC SYNTHESIS, R. E. Gawley and J. Aube, Eds., Elsevier Publishers: Amsterdam, (1996).

Other Publications

Higginbotham et al., Baseline Characteristics of Black and White Patients, OPHTHALMOLOGY 105(7):1137-1145 (1996).

Higginbotham et al., THE ADVANCED GLAUCOMA INTERVENTION STUDY (AGIS):3. Leske, M. C. et al., ARCH. OPHTHALMOL. 112:821-829 (1994).

Ohno, M. and Otsuka, M., ORGANIC REACTIONS, volume 37, page 1 (1989).

Sommer, A. et al., ARCH. OPHTHALMOL. 109:1090-1095 (1991).

Tielsch, J. M. et al., JAMA 266:369-374 (1991).

What is claimed is:

1. An improved method of treating glaucoma and ocular hypertension in a latent responding patient by topical ocular administration of a prostaglandin derivative or analog, the improvement comprising administering the prostaglandin derivative or analog at a dose which substantially exceeds the baseline dose for such prostaglandin analog to thereby achieve greater intraocular pressure reduction in such patient.

2. The method of claim 1 wherein the prostaglandin analog is selected from the group consisting of latanoprost, travoprost, unoprostone isopropyl, and bimatoprost.

3. The method of claim 2, wherein the prostaglandin analog is latanoprost, and wherein topical ocular administration is effected in an ophthalmically acceptable vehicle having a concentration of latanoprost exceeding 0.005%.

4. The method of claim 3, wherein the concentration of latanoprost is from 0.005% to 0.1%.

5. The method of claim 4, wherein the concentration of latanoprost is from 0.007% to 0.01%.

6. The method of claim 2, wherein the prostaglandin analog is travoprost and wherein the topical ocular administration is effected with an ophthalmically acceptable vehicle having a concentration of travoprost of at least 0.004%.

7. The method of claim 6, wherein a concentration of travoprost is from 0.004% to 0.01%.

8. The method of claim 2, wherein the prostaglandin analog is bimatoprost and wherein the topical ocular administration is effected with an ophthalmically acceptable vehicle having a concentration of bimatoprost in excess of 0.03%.

9. The method of claim 8, wherein the concentration of bimataprost is from 0.03% to 1%.

10. The method of claim 9, wherein the concentration of bimatoprost is from 0.05% to 0.1%.

11. A method of treating glaucoma or ocular hypertension in a latent responding patient, said method comprising administering travoprost to a patient, wherein the amount of travoprost administered is from 1 μg to 10 μg.

12. The method of claim 11, wherein the amount of travoprost administered is from 1.25 μg to 5 μg.

13. The method of claim 12, wherein the amount of travoprost administered is 2 μg.

14. The method of claim 11, wherein the travoprost is administered in a composition comprising 0.004% travoprost, and wherein at least 2 drops of the composition are administered to the patient, the second drop being administered within about five to ten minutes after the first drop and each subsequent drop being administered within about five to ten minutes of the previous drop.

15. A method of treating glaucoma or ocular hypertension in a latent responding patient, said method comprising administering latanoprost to a patient, wherein the amount of latanoprost administered is from 1.25 μg to 10 μg.

16. The method of claim 15, wherein the amount of latanoprost administered is from 1.25 μg to 5 μg.

17. The method of claim 16, wherein the amount of latanoprost administered is from 1.25 to 2 μg.

18. The method of claim 15, wherein the latanoprost is administered in a composition comprising 0.005% latanoprost, and wherein at least 2 drops of the composition are administered to the patient, the second drop being administered within about five to ten minutes after the first drop and each subsequent drop being administered within about five to ten minutes of the previous drop.

19. A method of treating glaucoma or ocular hypertension in a latent responding patient, said method comprising administering bimatoprost to a patient, wherein the amount of bimatoprost administered is from 9 μg to 30 μg.

20. The method of claim 19, wherein the amount of bimatoprost administered is from 10 μg to 15 μg.

21. The method of claim 20, wherein the amount of bimatoprost administered is from 11 μg to 14 μg.

22. The method of claim 19, wherein the bimatoprost is administered in a composition comprising 0.03% bimatoprost, and wherein at least 2 drops of the composition are administered to the patient, the second drop being administered within about five to ten minutes after the first drop and each subsequent drop being administered within about five to ten minutes of the previous drop.
